(19) United States
(12) Patent Application Publication
(10) Pub. No.: US 2008/0171085 A1
(43) Pub. Date: Jul. 17, 2008

(54) Novel biphasic delivery system for a pharmaceutical or nutraceutical composition and method of administration

(75) Inventors: Dahlia Elnekave, Northridge, CA (US); Gopi Mohan Sundaram, Encino, CA (US); Michael Todd Yatcilla, Los Angeles, CA (US)

Correspondence Address: Gabor L. Szekeres, P.O.Box 27938, Anaheim Hills, CA, 92809, US

(73) Assignee: Natrol, Inc.

(21) Appl. No.: 11/652,444

(22) Filed: Jan. 11, 2007

(51) Int. Cl. A61K 9/24; A61K 31/122; A61K 31/165; A61K 31/4045; A61K 31/4375; A61K 31/522; A61K 35/74; A61K 36/16; A61K 36/17; A61K 36/38; A61K 36/82; A61K 36/84; A61K 36/8962; A61K 38/44; A61K 38/45

(52) U.S. Cl. 424/465; 424/472; 424/725; 424/733; 424/752; 424/754; 424/93.46; 424/94.4; 424/94.5; 514/263.3; 514/283; 514/415; 514/627; 514/681

(57) ABSTRACT

A novel biphasic pharmaceutial or nutraceutical composition delivers both an immediate and a sustained dose of a pharmaceutical or nutraceutical agent in the same unit dose. An oral unit dose of melatonin includes melatonin in a sustained-release matrix contained in a solid phase, such as a tablet of capsule, and also includes melatonin in a film dispersed on the surface of the solid phase. Approximately 80 to 90 weight percent of the total amount of melatonin of each unit is contained in the sustained-release matrix and the remainder in the film. The novel composition permits a mammal, including a human being, to both rapidly fall asleep and remain asleep for a desired period of time by releasing an immediate-release dose and a sustained-release dose of melatonin.

### BACKGROUND OF THE INVENTION

1. Field of the Invention

The present invention is directed to a pharmaceutical or nutraceutical composition in a novel biphasic delivery system for administration to provide immediate and longer term pharmaceutical or nutraceutical benefit. More specifically, the present invention is directed to an example of the inventive composition having melatonin in a novel biphasic delivery system for its administration to induce immediate and prolonged sleep to a mammal, including a human being.

2. Brief Description of Prior Art

There are numerous pharmaceutical and nutraceutical agents known in the art that are likely to benefit from a manner of administration wherein a certain dose of the agent is administered quickly for immediate or virtually immediate absorption by the body to have an immediate or virtually immediate therapeutic effect, combined with another dose administered slower for longer term therapeutic effect.

Sleep is an essential component of good mental and physical health. Certain individuals are unable to obtain sufficient continuous, restorative sleep to maintain mental alertness and physical well-being. Sleep disturbances are present in over 74% of the adult population, and can stem from many sources. It is estimated that approximately one in four Americans suffer from regular sleep problems, according to the National Institutes of Health. (Patlak M. Your Guide To Healthy Sleep. Washington, D.C.: US Dept Health and Human Services; 2005. NIH Publication No. 06-5271. Available at: http://www.nih.gov.) Insomnia or the perception of inadequate or non-restful sleep was reported by over 52% of the respondents in the National Sleep Foundation 2005 Sleep in America Poll. (WB&A Market Research. National Sleep Foundation 2005 Sleep in America Poll. Available at: http://www.sleepfoundation.org.)

Research has shown that the lack of a good night's sleep is more harmful than previously thought. Health experts have shown that sleep is more than bodily rest. It has been found that the brain is very active during sleep and adequate sleep is necessary to overall health, safety, and performance. (National Institute of Health, supra.) Sleep plays a role in many aspects of daily life, including but not limited to, the ability to learn, create memories, solve problems and maintain a healthy mental outlook. Chronic lack of sleep can result in attention and memory problems, poor quality of life, depression and inability to focus and respond quickly. (Haack M, et al. Sustained sleep restriction reduces emotional and physical well being. Pain. 2005; 119:56-64; Kryger M, et al. Sleep, health and aging: bridging the gap between science and clinical practice. Geriatrics. 2004; 59:24-26, 29-30.) The prevalence of insomnia has been shown to be related to the age and sex of the individuals, being higher in older individuals and in females.

Early common treatments for insomnia have included depressants such as barbiturates, which are long acting and commonly became additive. They also include many unwanted side effects such as confusion, depression and sleep hangovers. Similar, although lessened effects, were found with the benzodiazepine hypnotic agents that followed. More recently the non-benzodiazepine compounds have shown an improved side effect profile. Concerns about grogginess are associated with the use of sedatives currently available for the treatment of sleeplessness. It is these properties that in fact can prohibit the usage of such treatments at any time other than prior to bedtime.

Melatonin (N-acetyl-5-methoxytryptamine) is a natural neurohormone hormone produced and secreted by the pineal gland. It appears to modulate a variety of neural and endocrine functions in the body including the control of circadian rhythm, an internal 24-hour time keeping system that plays an important role in when we fall asleep and when we wake up. (Zawilska J B, et al. Melatonin: from biochemistry to therapeutic applications. Pol J. Pharmacol. 1999; 51:3-23.) Normal melatonin cycles can be disrupted for many reasons, including when a person is exposed to excessive light in the evening or too little light during the daytime. (Lockley S W, et al. Relationship between napping and melatonin in the blind. J Biol Rhythms. 1997; 12:16-25.) Some individuals appear to produce low levels of melatonin, such as older people and persons with sleep disturbances. and in sleep disturbed patients. (Graham D, et al. Declining melatonin levels and older people: how old is old? Neuro Endocrinol Lett. 2004; 25:415-418; Brusco L I, et al. Effect of melatonin in selected populations of sleep-disturbed patients. Biol Signals Recept. 1999; 8:126-131.) The highest levels of melatonin are found in children age 5 and under, after which levels begin to decline. (Zhdanova I, et al. Melatonin: a sleep-promoting hormone. Sleep; 1997; 20:899-907.) However recent research also suggests that young, healthy individuals may also benefit from taking melatonin. (Pires M L N, et al. Acute effects of low doses of melatonin on the sleep of young healthy subjects. J Pineal Res. 2001; 31:326-332; Zhdanova I V, et al. Effects of low oral doses of melatonin, given 2-4 hours before habitual bedtime, on sleep in normal young humans. Sleep. 1996; 19:423-431.)

Thus, melatonin has been shown to hasten the onset of sleep, increase total sleep time, and improve sleep efficiency while reducing wakefulness. Plasma melatonin profiles display great intra-subject homogeneity and are highly reproducible from day to day in the same subject and thus represent one of the most robust measures of circadian rhythm and provide good indicators of melatonin secretion. Darkness stimulates its secretion and light suppresses its activity, including artificial light of sufficient intensity and duration administered at night. (Karasek M Melatonin in humans: where we are 40 years after its discovery humans. Neuro Endocrinol Lett. 1999:20:179-188.) Melatonin is lipid-soluble and released into the bloodstream and cerebrospinal fluid as it is synthesized. In the body, melatonin reinforces the nocturnal decrease of central temperature, an event that facilitates sleep propensity. In addition, melatonin has a modulatory effect on cortisol secretion.

Melatonin has several clear advantages over sleep drugs. As doses of currently recognized hypnotic agents are increased, increasing degrees of sleepiness and eventually coma results. In contrast, melatonin doses of several grams, given to humans, can raise blood levels to concentrations that are over 1,000 times physiological levels, but never produce involuntary loss of consciousness. (Waldhauser F, et al. Sleep laboratory investigations of hypnotic properties of melatonin. Psychopharmacology. 1990; 100:222-226.) Melatonin has a short biological half-life ranging from 32 to 48 minutes for 2 to 100 mg doses and is rapidly metabolized by the liver. (Aldhouse M, et al Plasma concentrations of melatonin in man following oral absorption of different preparations. Br J Clin Pharmacol. 1985; 19:517-521.) Doses of melatonin from very small to up to 10 grams have been administered to humans with no serious side effects. (Fitzpatrick A. Melatonin in health and disease. Altern Complement Ther. 2006; 12:282-291.) It has direct sedative/hypnotic properties in normal human subjects and investigations have shown that sleepiness occurs after administration. Most importantly, it appears that melatonin does not have the side effects associated with some hypnotics, including addiction, memory loss and “sleep hangover.” Further, melatonin is currently available in the United States, without a prescription.

In terms of difficulties in the sleep cycle itself, two factors are recognized. The first is difficulty initially falling asleep. The second is being reawakened in the middle of the sleep cycle and having difficulty returning to sleep. Many people suffer one or both phenomena in their sleep cycle. The following United States Patents and Published Applications for United States Patents disclose pharmaceutical compositions and methods including the use of melatonin for the purpose of treating insomnia, inducing sleep or steadying sleep patterns: U.S. Pat. Nos. 7,001,611; 6,818,665; 6,794,407; 6,703,412; 6,638,963; 6,620,836; 6,423,738; 5,707,652; 5,449,683; 6,129,930; U.S. Published patent applications Nos. 2005/027690; 2005/0164987. U.S. Pat. No. 6,129,930 discloses a composition that includes nicotinic acid in a hydroxypropylmethylcellulose (HPMC) matrix to treat hyperlipidemia at night.

Nevertheless, in spite of the above noted disclosures attempting to treat insomnia by administration of melatonin, there is currently still an unmet need for a melatonin product that affords relief for the symptoms of difficulty in falling asleep and difficulty in returning to sleep upon awakening during the sleep cycle. The primary example of the present invention, related to melatonin, addresses both of these factors via a dual-release (biphasic) melatonin composition that preferably has the physical form of a coated tablet.

Similar dual-release (biphasic) administration of other pharmaceutical or nutraceutical agents addresses problems of a similar nature, namely the need for rapidly administering a therapeutic dose of a pharmaceutical or nutraceutical agent for immediate effect followed by a slower administration of the same agent for a longer term, prolonged effect.

### SUMMARY OF THE INVENTION

The present invention relates to a novel pharmaceutical or nutraceutical composition that provides biphasic delivery capable of both immediately delivering therapeutic benefit of a pharmaceutical or nutraceutical agent, as well as providing sustained therapeutic benefit of the agent in either a human or an animal, comprising a pharmaceutical solid unit dose containing a therapeutic dose of the pharmaceutical or nutraceutical agent in a sustained-release matrix for sustained benefit coated with a film containing a dispersed form of a therapeutic dose of the pharmaceutical or nutraceutical agent for immediate or virtually immediate benefit. The present invention also relates to the method of treating a mammal, including a human being, in need of such treatment with the pharmaceutical or nutraceutical composition of the invention.

The presently most preferred embodiment of the invention relates to inducement of sleep and maintenance of a steady sleep pattern in a human being. It comprises a melatonin composition that combines a fast-release and a controlled-release delivery vehicle for a desired period of time. The composition releases melatonin immediately via a soluble melatonin film coating that produces immediate sleep onset, and subsequently releases melatonin slowly using a hydroxypropyl matrix over a period of hours, thereby sustaining the sleep cycle. Studies have shown that deficiencies in plasma melatonin concentrations associated with aging can be addressed with melatonin supplementation, which more specifically mitigates the sleep-wake cycle disturbances and circadian-based sleep imbalances. (Zeitzer, J M, et al. Do plasma melatonin concentrations decline with age? Am J Med 1999; 107:432.) Much like the initial concentration at the time of administration, which induces sleep, the continual delivery of melatonin would prevent the subsequent reawakening of the subject during the sleep cycle. The preferred embodiment is a tablet, consisting of a core having melatonin dispersed in a matrix of HPMC coated with an aqueous film that contains melatonin.

The aqueous film coating affects immediate release of melatonin as the film disintegrates immediately upon coming in contact with an aqueous environment (e.g., the human gastrointestinal tract) thus releasing a first dose of melatonin immediately upon ingestion of the tablet. This rapidly increases serum levels of melatonin and helps subjects to fall quickly asleep. Next, the HPMC matrix of the tablet core disintegrates very slowly in an aqueous environment, releasing melatonin slowly over a period of time. This increases serum levels of melatonin over a long period of time, helping subjects stay asleep.

By adjusting the levels of HPMC in the tablet core, one is able to adjust the release profile of the sustained-release phase of the biphasic delivery. This composition is capable of delivering an immediate dose and maintaining a therapeutic level of melatonin in the subject's circulatory system over a pre-selected period of 2, 4, 6 or 8 hours, depending on the percentage of HPMC, which controls the overall release profile of the melatonin to the subject for the period of sleep chosen. The composition is useful in the treatment of various sleep anomalies as well as an overall aid to sleeping in the event of normal agitation, stress or sleep disruption.

Thus, the present invention addresses the unrealized needs of the prior art by providing a novel biphasic delivery mechanism for a melatonin (or other) composition capable of providing both an immediate and a sustained/controlled (timed) dosing of melatonin enabling the subject to both fall asleep immediately and also stay asleep for the desired amount of time.

### DETAILED DESCRIPTION OF THE INVENTION, DESCRIPTION OF PREFERRED EMBODIMENTS

Generally speaking, there are numerous pharmaceutical and nutraceutical agents that can provide an immediate therapeutic benefit, usually of a shorter duration, but for which a prolonged therapeutic benefit is also desired. Examples of health and related issues that can be treated with such agents include insomnia (sleep), digestion (gastrointestinal health), nutrition and diet involving appetite suppressant and/or thermogenic agents, memory and brain function, mental and physical activity, alertness and sports, cardiovascular health and mood and mood swings. For each of these exemplary health and/or nutritional issues, the present invention provides a composition that includes a core containing an agent that is released gradually for a sustained effect and a coating containing the same or related agent in a dispersed form or a form otherwise suitable for quick release and immediate or virtually immediate absorption by the mammalian body.

The term pharmaceutical agent or nutraceutical agent in the present description includes a single chemical entity, such as melatonin for insomnia, caffeine for alertness, vitamin K for cardiovascular health, capsaicin for appetite suppression or thermogenic effect and NADH or ATP for sports nutrition. The term agent, as used in the present description, also includes enzymes and other active ingredients or combination of active ingredients such as amylase, proteases, lipase and related enzymes for digestion and gastrointestinal health, or P57AS3 (active ingredient of Hoodia gordoni) for appetite suppression. The term agent still further includes the pharmaceutically or nutritionally accepted source of the previously noted and related agents, such as Lactobacillus acidophilus and Lactobacillus bifidus for digestion, green tea for appetite suppression or thermogenic effect, Ginkgo biloba for improved memory and brain function, garlic for cardiac health and St. John's Wort for improving mood and/or reducing mood swings. It is emphasized that the foregoing is a non-limiting list of examples. Table 1 below conceptually and in broad sense shows the examples of the desired therapeutic or nutraceutical benefit (Application), the agent contained in the fast-release coating (Coating) and the agent contained in the sustained-released core (Core) of the novel compositions of the present invention.

TABLE 1

| Application | Coating | Core |
|---|---|---|
| Sleep | Melatonin | Melatonin |
| Digestion/Gastrointestinal | Digestive Enzymes | Probiotics |
| Health | (amylase, protease, | (Lactobacillus |
| | lipase) | acidophilus, |
| | | L. bifidus) |
| Diet: Appetite | P57AS3 (Hoodia | Green Tea/Caffeine |
| Suppressant/Thermogenic | gordonii active | Capsiate |
| | ingredient) | Capsaicin |
| | | Ephedra |
| Brain Health | Vinpocetine | Ginkgo biloba |
| Sports Nutrition | Caffeine | NADH and/or ATP |
| Heart Health | Vitamin K | Garlic |
| Mood | Valerian extract | St. John's Wort |

The agent contained in the core, designed for sustained release can generally be contained in a hard tablet, hard or soft gel capsule or any other generally accepted form of solid formulation. For sustained release, the agent in the core is usually and preferably admixed in a matrix of HPMC. It is well understood in the art that tablets, hard and soft gel capsules and other solid forms of formulations of drugs, vitamins and nutraceuticals usually include excipients such as sugar, starch, other forms of microcrystalline cellulose and the like, which per se are well known in the art and need not be further described here. It is also within the scope of the present invention to contain the agent in a tablet or capsule in admixture with the usual type of excipients, or in an admixture with such excipients and HPMC.

The agent contained in the coating can be formed by depositing the agent in an aqueous solution, or in a solution of other suitable volatile solvent on the surface of the solid core tablet or capsule. Examples of suitable volatile solvents, other than water, are ethyl alcohol, propyl alcohol or other volatile solvents that after full evaporation leave no toxic residue behind.

The amount or dose of the agent in the coating and in the core depends on the type of agent or agents and on the nature of the therapeutic effect being sought. In this regard, known doses of the respective agents for the respective therapeutic effects are applicable but may require such modifications that will be readily apparent to those skilled in the art, or may be arrived to by routine experimentation.

Table 2 below shows for certain exemplary applications the therapeutic effect being sought (Application), the type and dose range of agent in the coating (Coating), the type and dose range of agent in the core and the matrix in the core (Core), in accordance with the present invention.

TABLE 2

| | Coating | | | Core | | |
|---|---|---|---|---|---|---|
| | | Dose | | | Dose | |
| Application | Agent | Range | | Agent | Range | Matrix |
| Sleep | Melatonin | 0.1–2.0 | mg | Melatonin | 0.5–10 mg | HPMC |
| Digestion/ | Digestive | 0.1–10 | mg | Probiotics | 100–400 mg | HPMC |
| Gastrointestinal | Enzymes | | | (Lactobacillus | | |
| Health | (amylase, | | | acidophilus, | | |
| | protease, | | | L. bifidus) | | |
| | lipase) | | | | | |
| Diet: Appetite | P57AS3 | 0.1–5.0 | mg | Green | 50–500 mg | HPMC |
| Suppressant/ | (H. gordonii | | | Tea/Caffeine | green tea | |
| Thermogenic | active | | | | extract; | |
| | ingredient) | | | | 25–200 mg | |
| | | | | | caffeine | |
| Diet: Appetite | P57AS3 | 0.1–5.0 | mg | Capsiate | 5–50 mg | HPMC |
| Suppressant/ | (H. gordonii | | | | | |
| Thermogenic | active | | | | | |
| | ingredient) | | | | | |
| Diet: Appetite | P57AS3 | 0.1–5.0 | mg | Capsaicin | 5–50 mg | HPMC |
| Suppressant/ | (H. gordonii | | | | | |
| Thermogenic | active | | | | | |
| | ingredient) | | | | | |
| Diet: Appetite | P57AS3 | 0.1–5.0 | mg | Ephedra | 2–40 mg | HPMC |
| Suppressant/ | (H. gordonii | | | | ephedrine | |
| Thermogenic | active | | | | alkaloids | |
| | ingredient) | | | | | |
| Brain/Memory | Vinpocetine | 1–10 | mg | Ginkgo Biloba | 60–240 mg | HPMC |
| improvement | | | | extract | | |
| Sports | Caffeine | 1–20 | mg | NADH and/or | 1–20 mg | HPMC |
| Nutrition, | | | | ATP | NADH; | |
| Mental | | | | | 1–20 mg | |
| Alertness | | | | | ATP | |
| Cardiac Health | Vitamin K | 10–200 | mcg | Garlic | 200–1200 mg | HPMC |
| Mood | Valerian | 1–10 | mg | St. John's Wort | 50–400 mg | HPMC |
| | extract | | | | | |

A single tablet, capsule or other solid formulation in accordance with the invention can be referred to as a unit. The dose ranges in Table 2 above are written for a single unit. The number of units of the composition to be administered to an adult human being in a day, and the time(s) of administration of the unit(s), depends on the nature of the therapeutic effect to be sought and the nature of the composition. Again, general knowledge in the art pertaining to the use of the applicable pharmaceutical or nutraceutical agents provides initial guidance for the administration of the compositions of the present invention. By way of example, compositions of the invention to provide a health or related benefit are administered in accordance with the present invention as follows:

Compositions of the invention to provide an appetite suppressant/thermogenic benefit may be administered 1 to 3 times per day, one hour before a meal.
Compositions of the invention to provide a brain (memory improving) benefit may be administered 1 to 3 times a day with meals.
Compositions of the invention to provide a sports nutrition, mental alertness benefit may be administered daily, before exercise.
Compositions of the invention to provide a cardiovascular health benefit may be administered 1 to 3 times per day.
Compositions of the invention to provide a mood improvement benefit may be administered 1 to 3 times per day depending on which agent is used.

### MOST PREFERRED EMBODIMENTS

The most preferred embodiment of the present invention relates to a composition and method of providing a novel dual delivery (biphasic) system for oral administration of melatonin to both induce immediate sleep, as well as to prevent reawakening during the sleep cycle in a mammal, including a human being. This is achieved by delivering 0.5 to 10 mg of melatonin in a solid unit dose (either a hard-shell gelatin capsule or a tablet) where 80% to 90% of the melatonin is contained in the “interior” of the unit dose (in the capsule fill or in the tablet core) and 10% to 20% of the melatonin is contained in an external film that is applied to the solid unit dose.

The exterior of the unit dose consists of melatonin and an aqueous film coating consisting of water, a biopolymer or synthetic polymer, and a plasticizer. This provides immediate-release melatonin. The resins and plasticizers used in the aqueous film coating are the types normally used in pharmaceutical applications. Examples of such known polymers are: HPMC, hydroxypropylcellulose, methylcellulose, polyvinylpyrridone (PVP) phthalates and derivatives, methacrylic acid copolymer and shellac. Examples of such known plasticizers are: glycerine, propylene glycol, polyethylene glycol (PEG), triacetin and triethyl citrate.

The interior of the unit dose consists of melatonin, HPMC and other appropriate excipients known by one skilled in the art as appropriate for manufacture of a pharmaceutical solid oral unit dose. The amount of HPMC is chosen based on the desired release profile, and varies from 5% to 20% of the total formula weight. This provides sustained-release melatonin.

The compositions described herein are administered to a human or animal desiring immediate and sustained sleep by conventional routes of administration in any manner known to those skilled in the art. Preferably, the compositions are in tablet form for oral administration, in amounts therapeutically effective to produce sleep for the desired length of time and may be administered to a human or animal suffering from an irregular sleep pattern due to aging, anxiety, depression, physical activity, illness, or any change in sleeping habits, and may also be taken just prior to sleeping to ensure an unbroken sleep cycle. Other sources of sleep disturbance stem from disruptions of the normal day-night cycle such as shift work and jet lag, both of which disrupt the body's circadian rhythm.

The present invention aids with difficulties in initially falling asleep as well as staying asleep and/or achieving a restful restorative sleep providing an unmet need and an improvement on current sleep aids that address only one phase of the sleep cycle.

Melatonin is included in the compositions described herein based on historical safety and efficacy results such as those described above. However, the concentrations of melatonin in the compositions described herein may vary according to desired length of sleep, age, weight or other factors. The currently most preferred embodiment of the present invention is a tablet that delivers 0.5 to 10 mg of melatonin where 80% to 90% of the melatonin is contained in the “interior” of the unit dose (e.g., in the capsule fill or in the tablet core) and 10% to 20% of the melatonin is contained in an external film that is applied to the solid unit dose.

In this preferred embodiment of the invention, the exterior of the unit dose consists of melatonin, water, methylcellulose and glycerine. This exterior provides immediate-release melatonin. The interior of the unit dose consists of melatonin, HPMC and other appropriate excipients known by one skilled in the art as appropriate for manufacture of a tablet. These excipients include binders, lubricants, glidants and bulking agents, which per se are well known in the art and need not be described here. The amount of HPMC is chosen based on the desired release profile, and varies from 5% to 20% of the total formula weight. This provides sustained-release melatonin. As it will be readily understood by those skilled in the art, the greater the amount of HPMC in a tablet, the longer time it takes to release the pharmaceutical agent contained in the matrix.

In the just described preferred embodiment, the ingredients comprising the interior of the unit dose are blended using traditional dry powder blending techniques and compressed using traditional tablet compression methods. Separately, the ingredients comprising the exterior of the unit dose are dispersed in water using traditional film coating dispersion methods and applied to the tablet cores using traditional film coating methods.

In accordance with the preferred method of administration (treatment) of the present invention, the just described coated tablets are administered orally to humans desiring immediate and sustained sleep.

In the ensuing description and claims, certain components are disclosed in the possible range of percentages, and the maximum numbers of each range may exceed 100%. It should be understood that it is not the intention of the applicant to disclose compositions in which the total of the sum of the percentage of individual components exceeds 100%, which composition is incapable of existence. Therefore, it should be understood that when one ingredient or component is in a high range then the percentage range(s) of another component or components are necessarily reduced so that the total percentages do not exceed 100.

The ingredients or components in the just described preferred embodiments, having tablet cores weighing 100 to 500 mg each, are present in the following ranges:

Core:
Melatonin (0.1-9%)
Dicalcium phosphate dehydrate (15-40%)
Stearic acid (2.5-4%)
Magnesium stearate (2.5-4%)
Hydroxypropylmethylcellulose (5-20%), the amount chosen depending on the desired release profile
Microcrystalline cellulose, or similar excipient selected from the group consisting of modified starches, maltodextrin and ethyl cellulose (5-70%)

Coating:
Melatonin (0.001-0.02%)
Water (92-95%)
Methylcellulose (5-7%)
Glycerine (0.2-1%)

The tablets are made by weighing the ingredients, blending them in a cross-flow rotary blender for 10 to 30 minutes, and compressing the powdery mixture by using a rotary tablet press of standard design. The coating is prepared by blending glycerine and methylcellulose, dispersing the resultant blend into water using a vortex mixer, mixing under vortex for 30 to 60 minutes until the product is completely dispersed, adding the melatonin and mixing for an additional 15 to 60 minutes under vortex. The resulting solution is applied to the tablet cores by spraying the solution onto a rotating bed of tablet cores using a standard pharmaceutical tablet film coater, spraying until the tablets obtain a weight gain of 0.5% to 2%. The just described coated tablets are administered orally to humans desiring immediate and sustained sleep.

Actual examples for the preparation of the most preferred embodiments of the invention are provided below.

### EXAMPLE 1

The following ingredients were weighed:

| Number | Ingredient | Weight (g) |
|---|---|---|
| 1 | Melatonin | 22.5 g |
| 2 | Dicalcium phosphate dihydrate | 315.0 g |
| 3 | Stearic acid | 45.0 g |
| 4 | Silicon Dioxide | 31.5 g |
| 5 | Magnesium stearate | 45.0 g |
| 6 | Hydroxypropylmethylcellulose | 202.5 g |
| 7 | Microcrystalline cellulose | 688.5 g |

Ingredients 1, 2, 4, 6 and 7 were blended in a 1.5 cu. ft. cross-flow blender for 15 minutes.
Ingredients 3 and 5 were added and blended for 2 minutes. The resultant blend was fed into a rotary tablet press. Tablets were manufactured weighing 299 to 310 mg, with a hardness of 10.5 kP. These resultant tablet cores were set aside.
The following ingredients were weighed:

| Number | Ingredient | Weight (g) |
|---|---|---|
| 8 | Melatonin | 3.5 g |
| 9 | Water | 130.2 g |
| 10 | Methylcellulose | 8.82 g |
| 11 | Glycerine | 0.98 g |

Ingredient 9 was introduced into a vortex mixer and brought to vortex. Ingredients 10 and 11 were added to ingredient 9 and mixed under vortex for 30 minutes. Ingredient 8 was added and mixed under vortex for 30 minutes. This resultant coating solution was set aside.
The tablet cores were introduced into a laboratory coating system (Labcoat M) with a 15-inch fully perforated pan. The pan was rotated to about 15-17 rpm, with inlet air blowing across the tablet bed at 75° C., and exhaust air exiting the system at 45° C. The coating solution was introduced into this coating system and sprayed onto the tablet cores by pumping the solution using a peristaltic pump and plastic tubing. The spraying was achieved using an atomizing spray gun of standard design with an atomizing air pressure of about 30 psi and a spray rate of about 15 g per minute. Tablets were coated until the coating solution was completely sprayed. This manufacturing process resulted in 4,500 tablets. The resulting tablets had a weight of 302 to 314 mg, indicating the tablets gained 3 to 4 mg and had a distribution of the active agent melatonin as follows:

| Tablet Phase | Weight (mg) | Melatonin (mg) |
|---|---|---|
| Immediate release (coating) | 3 mg | 1 mg |
| Sustained release (core) | 299 mg | 4 mg |

These were tested for dissolution. The tablets had the following release profile:

| T = 15 min | T = 30 min | T = 1 hr | T = 2 hr | T = 4 hr | T = 8 hr |
|---|---|---|---|---|---|
| | 0.85 mg | 1.15 mg | | 2.0 mg | 2.8 mg |

T indicates time.

These tablets were administered to humans 20 minutes prior to bedtime.

### EXAMPLE 2

The following ingredients were weighed:

| Number | Ingredient | Weight (kg) |
|---|---|---|
| 1 | Melatonin | 2.25 kg |
| 2 | Dicalcium phosphate dihydrate | 35 kg |
| 3 | Stearic acid | 5 kg |
| 4 | Magnesium stearate | 5 kg |
| 5 | Silicon Dioxide | 3.5 kg |
| 6 | Hydroxypropylmethylcellulose | 15 kg |
| 7 | Microcrystalline cellulose | 85 kg |

Ingredients 1, 2, 5, 6, and 7 were blended in a 40 cu. ft. cross-flow blender for 15 minutes. Ingredients 3 and 4 were added and blended for 2 minutes. The resultant blend was fed into a rotary tablet press. Tablets were manufactured weighing 302 to 313 mg, with a hardness of 9 kP. These resultant tablet cores were set aside.
The following ingredients were weighed:

| Number | Ingredient | Weight (kg) |
|---|---|---|
| 8 | Melatonin | 0.5 kg |
| 9 | Water | 20 kg |
| 10 | Methylcellulose | 1.5 kg |
| 11 | Glycerine | 0.15 kg |

Ingredient 9 was introduced into a vortex mixer and brought to vortex. Ingredients 10 and 11 were added to ingredient 9 and mixed under vortex for 30 minutes. Ingredient 8 was added and mixed under vortex for 30 minutes. This resultant coating solution was set aside.
The tablet cores were introduced into a laboratory coating system (Hi Coater) with a 40-inch partially perforated pan. The pan was rotated at 16 rpm, with inlet air blowing across the tablet bed at 75° C., and exhaust air exiting the system at 45° C. The coating solution was introduced into this coating system and sprayed onto the tablet cores by pumping the solution using a peristaltic pump and plastic tubing. The spraying was achieved using an atomizing spray gun of standard design with an atomizing air pressure of 170 to 200 psi and a spray rate of 250 to −300 g per minute. Tablets were coated until the coating solution was completely sprayed. This manufacturing process produced 500,000 tablets. The resulting tablets had a weight of 306 to 318 mg, indicating the tablets gained 4 to 5 mg and had a distribution of the active agent melatonin as follows:

| Tablet Phase | Weight (mg) | Melatonin (mg) |
|---|---|---|
| Immediate release (coating) | 4 mg | 1 mg |
| Sustained release (core) | 302 mg | 4 mg |

These tablets were tested for dissolution. The tablets had the following release profile:

| T = 15 min | T = 30 min | T = 1 hr | T = 2 hr | T = 4 hr | T = 8 hr |
|---|---|---|---|---|---|
| 1 mg | 1.5 mg | 2 mg | 3 mg | 4 mg | 5 mg |

T indicates time.

These tablets were administered to humans 20 minutes prior to bedtime.

The foregoing description is provided for describing and disclosing various and preferred embodiments of the present invention in its general terms, describing the best mode and preferred embodiments. It should be understood that numerous modifications or alterations may be made by those skilled in the art on the basis of the present disclosure and without departing from the scope of the invention.

1. A biphasic composition having one or more agents selected from a group consisting of pharmaceutical and nutraceutical agents, the composition capable of immediate and also of sustained release of the agent, the composition comprising:
a first solid phase comprising a sustained-release matrix containing the agent for sustained release, and
a second water soluble phase forming a layer on the surface of the first solid phase and containing the agent in a dispersed form for immediate release, said water soluble phase further comprising water, a plasticizer and a polymer selected from the group consisting of a biopolymer and a synthetic polymer.

2. A composition in accordance with claim 1 wherein the agent is a substance capable of treating gastrointestinal problems or an agent for maintaining or improving digestion.

3. A composition in accordance with claim 2 wherein the agent in the first phase is probiotic bacillus and the agent in the second phase is a digestive enzyme.

4. A composition in accordance with claim 1 wherein the agent is an appetite suppressant.

5. A composition in accordance with claim 4 wherein the agent in the first phase is selected from a group consisting of green tea extract, caffeine, capsiate, capsaicin and ephedra and the agent in the second phase is PS57AS3.

6. A composition in accordance with claim 1 wherein the agent is a substance capable of maintaining or enhancing memory.

7. A composition in accordance with claim 6 wherein the agent in the first phase is Ginkgo biloba and the agent in the second phase is vinpocetine.

8. A composition in accordance with claim 1 wherein the agent is a substance capable of maintaining or increasing alertness.

9. A composition in accordance with claim 8 wherein the agent in the first phase is selected from a group consisting of NADH and ATP and the agent in the second phase is caffeine.

10. A composition in accordance with claim 1 wherein the agent is a substance for treatment of cardiovascular problems or a substance for maintaining or increasing cardiovascular health.

11. A composition in accordance with claim 10 wherein the agent in the first phase is garlic or garlic extract and the agent in the second phase is vitamin K.

12. A composition in accordance with claim 1 wherein the agent is capable of maintaining or enhancing mood.

13. A composition in accordance with claim 12 wherein the agent in the first phase is St. John's Wort and the agent in the second phase is valerian extract.

14. A biphasic composition of melatonin capable of quickly releasing a dose of melatonin for acting fast to induce sleep in a mammal, and also capable of slowly releasing melatonin for prolonged effect of sustaining sleep in the mammal, the composition comprising:
a first solid phase comprising a sustained-release matrix containing the melatonin for sustained release, and
a second water soluble phase forming a layer on the surface of the first solid phase and containing the melatonin in a dispersed form for immediate release, said water soluble phase furher comprising water, a plasticizer and a polymer selected from the group consisting of a biopolymer and a synthetic polymer.

15. A composition in accordance with claim 14 wherein the first solid phase is a tablet, the sustained-release matrix is hydroxypropylmethylcellulose containing a first quantity of melatonin, said matrix being contained in the tablet, and wherein the second phase is a film disposed on the surface of the tablet, the film containing a second quantity of melatonin.

16. A composition in accordance with claim 15 wherein the first phase contains 80 to 90 weight percent of the total amount of melatonin contained in the composition, and wherein the second phase contains 10 to 20 weight percent of the total amount of melatonin contained in the composition.

17. A composition in accordance with claim 15 wherein a single unit of the composition is a tablet having a total weight of 105 to 575 mg, and wherein the first phase comprises:
80 to 90 weight percent of the total amount of melatonin contained in the composition;
dicalcium phosphate dehydrate being 15 to 40 weight percent of the first phase;
stearic acid being 2.5 to 4 weight percent of the first phase;
magnesium stearate being 2.5 to 4 weight percent of the first phase; hydroxypropylmethylcellulose being 5 to 20 weight percent of the first phase, and
an excipient selected from the group consisting of microcrystalline cellulose, modified starches, maltodextrin and ethyl cellulose said excipient being 5 to 70 weight percent of the first phase;
the second water soluble phase is a film of 5 to 75 mg total weight, disposed on the surface of the first phase and comprises:
melatonin, said quantity being 10 to 20 weight percent of the total amount of melatonin contained in the composition, and
methylcellulose, glycerol and water.

18. A composition in accordance with claim 14 wherein the sustained-release matrix of the first phase comprises 1 to 5 weight percent hydroxypropylmethylcellulose whereby in a human being the release of melatonin results in a steady therapeutic level of approximately one (1) to three (3) hours.

19. A composition in accordance with claim 14 wherein the sustained-release matrix of the first phase comprises 3 to 8 weight percent hydroxypropylmethylcellulose whereby in a human being the release of melatonin results in a steady therapeutic level of approximately three (3) to five (5) hours.

20. A composition in accordance with claim 14 wherein the sustained-release matrix of the first phase comprises 5 to 10 weight percent hydroxypropylmethylcellulose whereby in a human being the release of melatonin results in a steady therapeutic level of approximately seven (7) to nine (9) hours.

21. A method of treating a human or animal subject, comprising: orally administering the composition of claim 14 approximately twenty (20) minutes prior to desired sleep time.
